(19) United States
(12) Patent Application Publication
(10) Pub. No.: US 2014/0235994 A1
(43) Pub. Date: Aug. 21, 2014

(54) OPTIMIZED VELOCITY-SELECTIVE ARTERIAL SPIN LABELLING MODULE

(71) Applicant: ISIS INNOVATION LTD., Oxford (GB)

(72) Inventors: Peter Jezzard, Oxford (GB); James Meakin, Oxford (GB)

(73) Assignee: ISIS INNOVATION LTD., Oxford (GB)

(21) Appl. No.: 14/020,335

(22) Filed: Sep. 6, 2013

Related U.S. Application Data
(60) Provisional application No. 61/697,939, filed on Sep. 7, 2012.

(51) Int. Cl. A61B 5/055; G01R 33/563

(52) U.S. Cl. CPC A61B 5/055; G01R 33/563
USPC 600/410

(57) ABSTRACT

A velocity selective preparation method is disclosed, for velocity selective arterial spin labelling (VSASL), the VSASL method using non-selective radiofrequency pulses and magnetic field gradients to modulate the longitudinal magnetization of the spins as a function of their velocity, wherein said velocity selective preparation method comprises an n-segment B₁ insensitive rotation that is resistant to eddy current artifacts.

### TECHNICAL FIELD

The present invention generally relates to measuring blood perfusion and, more particularly, relates to an Arterial Spin Labeling method for measuring perfusion in areas of slow or collateral blood flow, facilitating clinicians in making diagnostic, prognostic or therapeutic decisions. The present invention is particularly suitable, but not limited, to use where a patient has suffered a stroke, for examining gray or white matter in the brain, or for assessment of skeletal muscle.

### BACKGROUND

Arterial Spin Labeling (ASL) uses endogenous blood water as a freely diffusible tracer to noninvasively quantify perfusion. Classical techniques including pulsed and continuous ASL invert spins upstream to the imaging volume and then image spins that have exchanged into tissue. The necessary spatial separation between tagging and imaging regions can result in long bolus arrival times, which is one of the largest sources of error in the quantification of ASL. This is especially problematic in situations where bolus arrival time is already increased, such as stroke, white matter or skeletal muscle, leading to decreased signal-to-noise ratio or erroneous perfusion values.

Velocity-Selective ASL (VSASL) is a variant of pulsed ASL that eliminates the bolus arrival time by labelling the blood much closer to the tissue bed. VSASL uses non-selective radiofrequency (RF) pulses and magnetic field gradients to modulate the longitudinal magnetization of the spins (M_{z}) as a function of their velocity. The velocity-selective (VS) preparation saturates spins above a certain cut-off velocity (V_{c}), which are then imaged after they have exchanged into tissue. Through setting V_{c} to a value corresponding to the blood velocity at the arteriole-capillary bed interface the technique is made insensitive to bolus arrival time, as the tag is being generated within the imaging volume itself.

Several artefacts hinder accurate quantification of VSASL. B₁ and B₀ inhomogeneities lead to a mis-estimation of perfusion due to spatial variations in tagging efficiency. Additionally, the standard Double Refocused Hyperbolic Secant (DRHS) and order-4 B₁-insensitive rotation (BIR-4) VSASL sequences are not eddy current balanced.

Accordingly there is a need to address the aforementioned deficiencies. The aim of the present invention is therefore to provide a method that overcomes the deficiencies named above. The present invention is a velocity selective preparation to reduce eddy current effects. The present invention is robust to eddy currents and therefore improves the quality and reliability of VSASL measurements.

### SUMMARY

In an embodiment there is provided a velocity selective preparation, for velocity selective arterial spin labelling (VSASL) method, said VSASL method using non-selective radiofrequency pulses and magnetic field gradients to modulate the longitudinal magnetization of the spins as a function of their velocity, wherein said velocity selective preparation method comprises an n-segment B₁ insensitive rotation.

Other aspects of the invention are as laid out in the appended claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A and 1B depicts the radio frequency (RF) amplitude, phase and desired VS gradient waveform for the method of the present invention.

FIG. 2A depicts the simulated longitudinal magnetization of moving spins with arterial T₁ and T₂ as a function of B₀ off-resonance after the application of the VS preparation of the present invention, along with plots of the perturbations to the static spin magnetization caused by eddy currents of different time constant as a function of distance from isocenter. FIG. 2B depicts the response of static spins to the VS preparation.

FIG. 3 depicts representative brain perfusion maps from one subject acquired with the VS preparation of the present invention. The top image was acquired with G_{MAX}=10 mT/m and the middle image was acquired with G_{MAX}=40 mT/m. The bottom image shows good subtraction, as expected.

FIG. 4 depicts the correlation of the change in perfusion from the individual subject's mean perfusion with Gₘₐₓ for the preparation of the present invention.

FIG. 5A shows the symmetric BIR-8 pulse diagram, for V_{cut}−=2 cm/s at Gₘₐₓ=40 mT/m. FIG. 5B shows the expected BIR-8 eddy current sensitivity. FIG. 5C shows the expected symmetric BIR-8 eddy current sensitivity.

FIGS. 6A-6I depicts the application of BIR-4, BIR-8 and symBIR-8 in a silicone oil phantom at isocenter. FIG. 6A, FIG. 6D and FIG. 6G depicts the application of BIR-4 in a silicone oil phantom at isocenter. FIG. 6B, FIG. 6E and FIG. 6H depicts the application of BIR-8 in a silicone oil phantom at isocenter. FIG. 6C, FIG. 6F and FIG. 6I depicts the application of symBIR-8 in a silicone oil phantom at isocenter

FIGS. 7A-7I illustrate the subtraction errors in a silicone oil phantom for the BIR-4 (dotted line), BIR-8 (dashed line), and symBIR8 (solid line) preparation. FIGS. 7A-7C illustrate the subtractions wherein velocity encoding gradients are applied in X direction. FIGS. 7D-7F illustrate the subtractions wherein velocity encoding gradients are applied in Y direction. FIGS. 7G-7I illustrate the subtractions wherein velocity encoding gradients are applied in Z direction.

### DETAILED DESCRIPTION

Having summarized various aspects of the present disclosure, reference will now be made in detail to the description of the disclosure as illustrated in the drawings. While the disclosure will be described in connection with these drawings, there is no intent to limit it to the embodiment or embodiments disclosed herein. On the contrary, the intent is to cover all alternatives, modifications and equivalents included within the spirit and scope of the disclosure as defined by the appended claims.

### Velocity Selective Preparations

In the present method arterial spins are tagged based on their velocity rather than their spatial location. The velocity spin preparations saturate spins above a pre-defined V_{c}. The spins are first tipped into the transverse plane without spatial selection. Bipolar gradients are then applied which result in a phase accrual of the spins that is proportional to their velocity. The spins are then flipped back to the longitudinal axis. The longitudinal magnetization of the spins at the end of a VS preparation is then given by

M_{Z}(v)=Mₒα cos(γm₁v) (1)

where α is the tagging efficiency of the preparation, m₁ is the first moment of the VS gradients and v is the velocity of the spins. Within a laminar vessel the total expected magnetization is given by

$${M}_{z} ( {V}_{MAX} ) = \frac{{M}_{0} α}{{V}_{MAX}} {\int }_{0}^{{V}_{MAX}} cos ( γ {m}_{1} v ) d v = {M}_{0} α sinc ( γ {m}_{1} {V}_{MAX} ) \qquad ( 2 )$$

where V_{MAX} is the maximum velocity of the vessel. V_{c} is then defined as the first zero crossing of the sin c function, where V_{c}=π/(γm₁), above which the spins are considered to be saturated. It has previously been shown that for V_{c}<4 cm/s VSASL becomes insensitive to transit time in gray matter, therefore, in the present method V_{c}=2 cm/s is used. The cut off velocity can be in the range 0<|V_{c}|<=infinity.

To overcome the spatial variation in tagging efficiency of prior art methods, in the present invention, spins are in the transverse plane at the zero points of the RF amplitude function, so monopolar gradients for velocity selection are inserted between segments 1 and 2, and between segments 3 and 4, resulting in a spatially independent tagging efficiency.

In VSASL two acquisitions are made, a tag acquisition with m₁=π/(γV_{c}), and a control acquisition with m₁=0. Eddy currents generated by the VS preparation in the tag acquisition are not present in the control. To include the bipolar gradient concept to the improved B₀ and B₁ insensitive BIR preparation, the present invention uses an eight-segment B1 insensitive (BIR-8) VS preparation.

A BIR pulse produces an adiabatic rotation over a designed off-resonance range. The RF amplitude function (A(t)) is given by

$$A ( t ) = \{ \begin{matrix}tanh [ ζ ( 1 - \frac{t}{{T}_{SEG}} ) ] & 0 \leq t < {T}_{SEG} , odd segments \\ tanh [ ζ ( \frac{t}{{T}_{SEG}} ) ] & 0 < t \leq {T}_{SEG} , even segments\end{matrix} \qquad ( 3 )$$

where ξ is a dimensionless constant and T_{SEG} is the duration of one pulse segment. The corresponding phase is given by

$$φ ( t ) = \{ \begin{matrix}- {ω}_{MAX} {T}_{SEG} ln [ \frac{\vert cos ( κ \frac{t}{{T}_{SEG}} ) \vert}{κ tan ( κ )} ] & 0 \leq t < {T}_{SEG} , odd segments \\ - {ω}_{MAX} {T}_{SEG} ln \{ \frac{\vert cos ( κ \frac{t}{{T}_{SEG}} - 1 ) \vert}{κ tan ( κ )} \} & 0 < t \leq {T}_{SEG} , even segments\end{matrix} \qquad ( 4 )$$

where κ is a dimensionless constant and ω_{MAX} is the maximum frequency sweep. It can be shown that a composite BIR pulse made up of four segments is the most robust pulse to B₀ and B₁ inhomogeneity due to the time symmetry of φ(t) about the mid point of A(t).

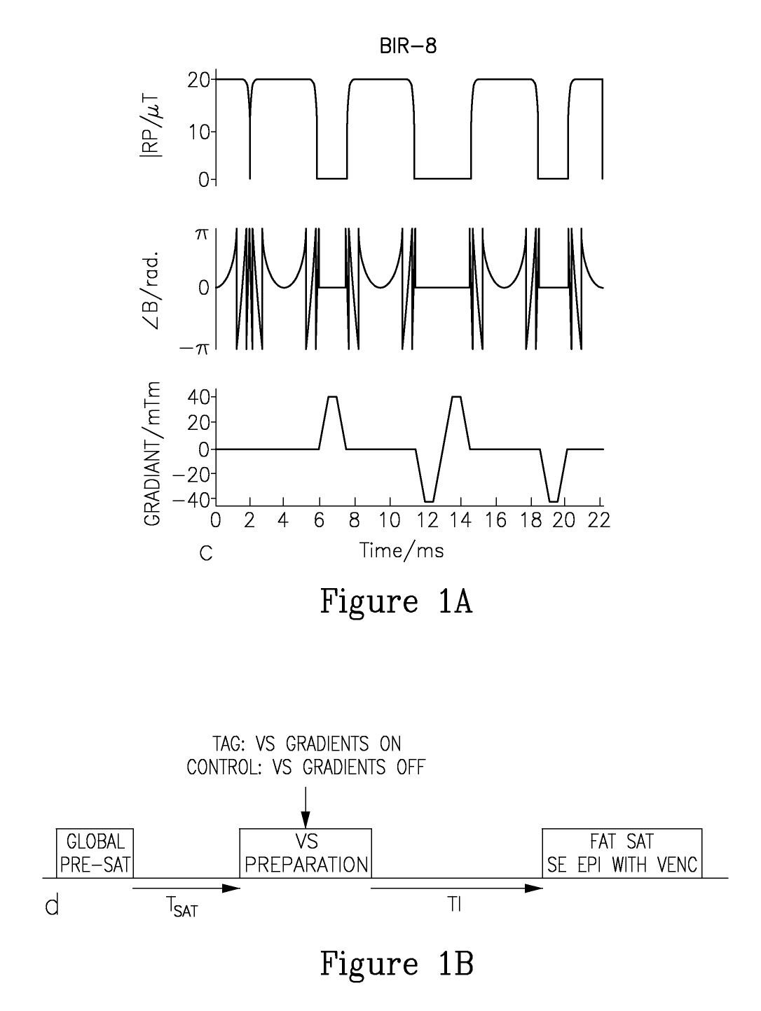

To allow for the addition of bipolar gradients, in the present method, the number of RF segments are eight. This maintains RF pulse symmetry about the mid point of A(t), and therefore preserves the B₀ insensitivity of the preparation. Of course, the number of RF segments may be more or less than eight. The present use of eight segments is an example only. The bipolar gradient lobes for velocity selection were then inserted between segments 3 and 4, segments 5 and 6, and between segments 7 and 8, where A(t)=0. FIG. 1A shows the RF amplitude (top), the RF phase (middle) and the desired VS gradient waveform (bottom). The gradients shown in the tag condition for V_{c}=2 cm/s, Gₘₐₓ=40 mT/m and gradient ramp time r=0.5 ms. In the control acquisition the gradients in the VS preparation are set to zero. The VS preparations were inserted into the pulse sequence (FIG. 1B), keeping Tₛₐₜ and TI constant. The gradients are arranged with timing ratios 0:1:2:1 at the A(t)=0 points of the BIR-8. This timing scheme balances the linear phase accrual from off resonance as the odd and even delays have the same total time. Subject to these constraints, any gradient pattern could be used within the BIR-8 preparation.

For the present method, the BIR-8 preparation is designed so that the pulses are insensitive over ΔB₀=±250 Hz with an adiabatic threshold of 15 μT by optimising ξ, κ and ω_{MAX} through Bloch equation simulations. To limit the duration of the preparations, T_{SEG}=2 ms, is set. However, it will be understood by the skilled person that other values may be used.

### Bloch Equation Simulations

A Bloch equation simulation was used to evaluate the responses of the VS preparation to the presence of B₁ and B₀ inhomogeneity and eddy currents. The simulation considers rotations about the effective B field followed by relaxation with a time step of 5 μs. The simulation was implemented in MATLAB 2011a (The MathWorks Inc., Natick, Mass., USA). However, any other suitable software may be used.

To determine the adiabatic threshold and off-resonance sensitivity of each preparation, the response of arterial spins were simulated. Simulations were performed over a range of ΔB₀ (±500 Hz), B₁ (0.5-25 μT) and v (−4-4 cm/s). A maximum gradient strength of 40 mT/m with a rise time of 0.5 ms was assumed. The predicted tagging efficiency for the preparation was also determined by simulation. As adiabatic pulses are used the relaxation decay of the bolus during the VS preparations is a mix of T₁ and T₂ effects. For the preparation of the present invention, the response of arterial spins with B₁=20 μT, v=0 and 2 cm/s were assumed, assuming arterial T₁=1664 ms (19) and T₂=150 ms (20). The tagging efficiency, a, for the preparation is then given by

$$α = \frac{{M}_{z} ( v = 0 ) - {M}_{z} ( v = 2 )}{2} \qquad ( 5 )$$

at the end of the preparation.

The effect of the preparation on static spins in the presence of eddy currents was also modelled. The eddy current effects are modeled as linearly independent components with eddy current amplitudes Aₙ and time constants τₙ. The additional gradient due to eddy currents (g(t)) is given by

$$g ( t ) = - \frac{\partial G ( t )}{\partial t} ⊗ {\sum }_{n}^{ } H ( t ) {A}_{n} exp ( - t / {τ}_{n} ) \qquad ( 6 )$$

where \textcircled{x} represents convolution with the desired gradient waveform, G(t), and H(t) is the unit step function. Then the static spins were simulated at different positions from gradient isocenter (±25 cm) with τₙ=10⁻⁴−1 s and Aₙ=0.001−1%. Only the presence of a single time constant τₙ was considered and relaxation effects were ignored.

### In Vivo Measurements

VSASL measurements with the preparation were performed in five healthy volunteers using a 3 Tesla Siemens Verio scanner (Siemens Healthcare, Erlangen, Germany) to assess the influence of eddy currents. The VSASL pulse sequence (FIG. 1B) begins with a global pre-saturation (22) to remove any spin history effects as the tag is being generated within the imaging volume. After time T_{SAT}=3.2 s the VS preparation is applied with V_{c}=2 cm/s. The tagging gradients were applied on the x axis, although other axes could be used. A spin echo, Echo Planar Imaging (EPI) readout is then applied after inflow time TI. During the readout portion of the sequence flow-crushing Stejskal-Tanner gradients with m₁=π/(γV_{c}) are applied for both tag and control acquisitions, on the same axis as the tagging gradient. This dephases spins above V_{c} so that only signal from spins that have exchanged into tissue during time TI, and thus have decelerated to a velocity below V_{c}, are acquired.

Other acquisition parameters were TR=5.1 s, TE=32 ms, TI=0.7 s, acquisition time per slice=61.92 ms, 18 slices, 256 mm FOV, 64×64 matrix, slice thickness=5 mm. The volunteers were moved so that the center of the imaging slice group was at the magnet isocenter. The VS preparations were played out on a whole body transmission coil at maximum amplitude (23 μT) and a 32-channel head receive coil was used. A separate body coil receive image was acquired for coil sensitivity correction and M₀^{CSF} calibration. A double inversion recovery acquisition with inversion times designed to null white matter and CSF was used as a gray matter mask with adiabatic inversions 4150 ms and 550 ms before an identical SE-EPI readout.

### Modulation of Eddy Currents In Vivo

The eddy current spectrum will be different for each scanner. As Aₙ and τₙ are generally not known, the eddy current amplitudes are varied by varying G_{MAX}. At the end of a gradient ramp (t=r), the unwanted additional gradient due to eddy currents is given by

$$g ( t = r ) = - {G}_{MAX} \cdot {\sum }_{n}^{ } \frac{{A}_{n} {τ}_{n}}{r} ( 1 - exp [ - t / {τ}_{n} ] ) \qquad ( 7 )$$

where G_{MAX} is the maximum amplitude of the desired trapezoidal gradient. Therefore, the eddy current gradient amplitude can be linearly modulated by applying the VS preparation with different G_{MAX}, keeping rise time r constant. For the preparation, five G_{MAX} values (10-40 mT/m) with r=0.5 ms were applied. Sixteen tag-control pairs were acquired for the preparation and G_{MAX} combination. The acquisition order was randomized. Total scan time was 50 minutes.

### Data Analysis

Data were corrected for motion and registered to the M₀ scan using FLIRT. Images were subtracted pairwise and then averaged to form the ΔM image. Perfusion was quantified on a voxelwise basis by non-linear fitting to a modified general kinetic model:

$$Δ M = {M}_{0}^{BLOOD} \cdot α \cdot f \cdot TI \cdot {q}_{p} ( f ) \cdot ( 1 - exp [ \frac{- {T}_{SAT}}{{T}_{1}^{BLOOD}} ] ) \cdot exp ( \frac{- TI}{{T}_{1}^{BLOOD}} ) \qquad ( 8 )$$

where M₀^{BLOOD} is the magnetization of a fully relaxed voxel of blood as determined from calculation via the M₀^{CSF} scan; α is the tagging efficiency of the VS preparation; ƒ is perfusion and qₚ(f), takes into account the different relaxation times of the bolus and the tissue. The quantification assumes that the bolus arrival time is zero and that the bolus length is equal to TI. Since reducing G_{MAX} will increase the tagging gradient duration, a for each VS preparation and G_{MAX} was simulated.

Mean perfusion, f(G_{MAX}), was calculated for each preparation and G_{MAX} within the gray matter mask derived from the subject's double inversion recovery scan. The effect of eddy currents on apparent perfusion should only depend on the scanner used, the relaxation times of static tissue and the TI, but not the underlying perfusion of an individual subject. Therefore, Δƒ=ƒ(G_{MAX})− ƒ̅(G_{MAX}) where ƒ̅(G_{MAX}) is the individual subject's perfusion, were correlated averaged over all G_{MAX} (reported in Table 1).

### Simulations

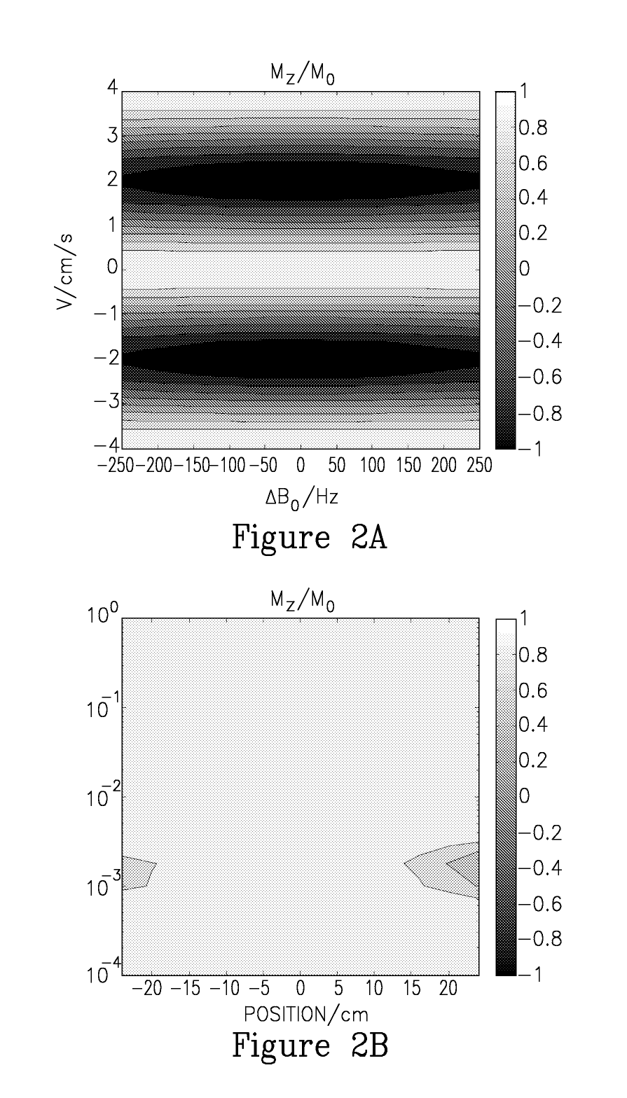

For the BIR VS preparation it was found that ξ=15, tan(κ)=60 and ω_{MAX}=39.8 kHz produced an adiabatic rotation over ΔB₀=±250 Hz. The adiabatic threshold was found as B₁=14 μT. FIG. 2A (top) shows the resulting longitudinal magnetization of moving spins after the application of the BIR-8 VS preparation with V_{c}=2 cm/s and G_{MAX}=40 mT/m. For on-resonant spins the tagging efficiency was found as α_{DRHS}=0.92, α_{BIR-4}=0.93, α_{BIR-8}=0.89. The simulations demonstrate that the desired co-sinusoidal modulation of magnetization as a function of velocity is produced for the preparation.

FIG. 2B (bottom) shows the predicted response of static spins to the VS preparation with G_{MAX}=40 mT/m and Aₙ=0.25%. At isocenter static spins are returned to +M_{Z}, as expected for a VS preparation. Simulations show the BIR-8 preparation of the present invention has very little sensitivity to eddy currents compared with DRHS and BIR-4 preparations.

### In Vivo Measurements

Mean gray matter perfusion values averaged over all G_{MAX} for the BIR-8 preparation are reported in Table 1, (below) corrected for differences in the theoretical efficiency for the preparation, and for regional receive coil sensitivity differences. The mean perfusion over all subjects for the BIR-8 preparation was 53.9±2.6 ml/100 g/min.

| Subject | Mean perfusion over all Gₘₐₓ (mL/100 g/min) |
|---|---|
| A | 45.9 ± 3.1 |
| B | 50.0 ± 2.2 |
| C | 59.7 ± 2.2 |
| D | 58.5 ± 1.6 |
| E | 55.8 ± 2.2 |
| Mean | 53.9 ± 2.6 |

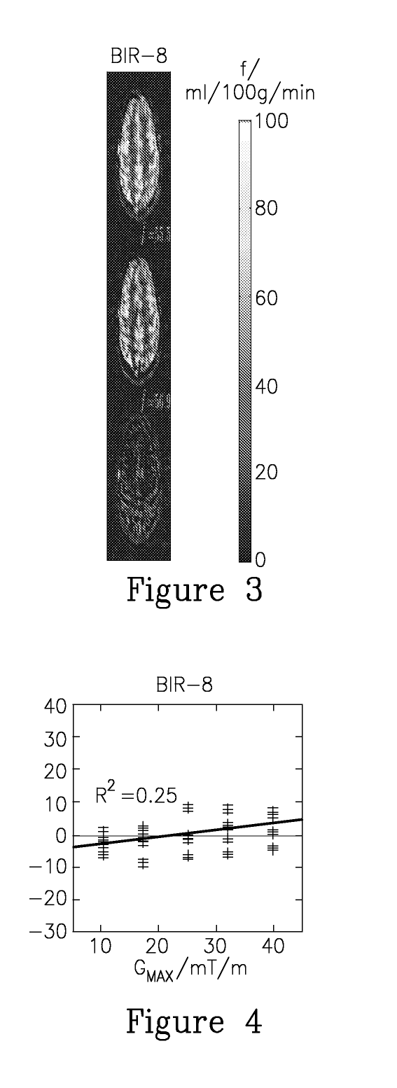

Representative perfusion maps are displayed in FIG. 3 for G_{MAX}=10 and 40 mT/m. The perfusion maps based on the BIR-8 preparation display reduced eddy current artifacts in the subtraction image compared with the DRHS and BIR-8 preparations.

FIG. 4 shows the variation of apparent perfusion versus G_{MAX} for the preparation. Perfusion measured by the BIR-8 method correlates to P=0.011. The slope of Δf/G_{MAX} was 0.21 (ml/100 g/min)/(mT/m) for the BIR-8 preparation. This compares favorably to the DRHS and BIR-4 preparations that show a greater dependence of perfusion with G_{MAX} value.

It has been shown that the BIR-8 VS preparation of the present invention is less sensitive to eddy-current effects, whilst preserving a good insensitivity to B₀ and B₁ inhomogeneities. The data show that the standard VS preparations may overestimate perfusion due to static spin contamination in the ΔM image, caused by eddy currents, but also shows that the BIR-8 preparation performs extremely well.

For the BIR-8 VS preparation the average gray matter perfusion estimates that were calculated over all G_{MAX} values fall within expected normal physiological ranges. For G_{MAX}=10 mT/m, the apparent perfusion as measured by the present invention was 51.4±3 ml/100 g/min.

The τₙ compensated by the preparation will depend on the time between the gradient lobes and the gradient rise time. Although changing G_{MAX} from 10 mT/m to 40 mT/m will change the time between the gradient lobes, simulations suggests that this would not significantly alter the τₙ distribution. In the present method, all the gradient durations within an individual VS preparation were equal for simplicity. The duration of the gradient lobes could be adjusted to null a particular τₙ, similar to the approach used for designing diffusion gradient, subject to the timing constraints of the BIR-8 pulse.

In the present case, the tagging gradients were applied on the x axis, since any changes in perfusion as a function of z slice position could be attributed to a slice timing error, which would cause an erroneous TI for each slice. The V_{c} of 2 cm/s means that the method may be sensitive to vessels on the order of arterioles in the cortical surface, so the direction of the encoding should not matter.

Although the BIR-8 preparation is RF intensive, SAR did not present a problem at 3 T with the protocol used. It was found that a TR of 2 s is possible, but will reduce the SNR due to a shorter T_{SAT}. To maximize SNR efficiency the TR and TI were chosen by maximizing ΔM/√TR (equation 8) for the central slice, with an expected perfusion of 60 ml/100 g/min. Equation 8 assumes that the bolus was in the field of view of the RF coil as the saturation pulse was played out, which may not be the case with the long TR used.

Further Eddy Current Reduction: symBIR-8

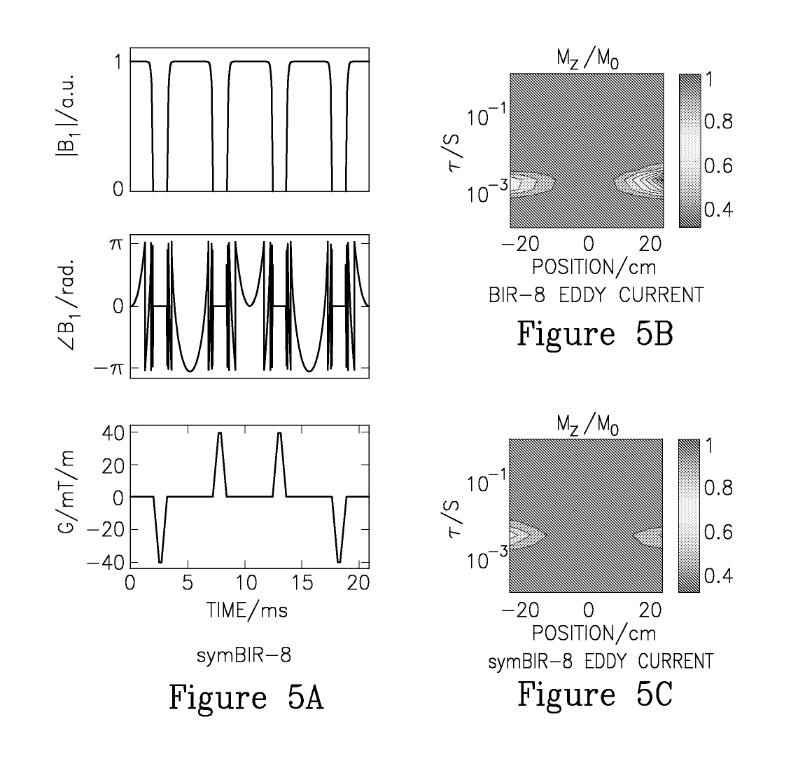

A further improvement of the BIR-8 method is the symBIR-8 method described in detail below. Here it is shown that the errors due to eddy currents can be further reduced by inserting gradient lobes at all four |B1|=0 points of the BIR-8 preparation with polarities −1:+1:+1:−1 (FIG. 5A). This symmetric preparation, symBIR-8 was implemented on the system and compared to the BIR-4 and BIR-8 preparations.

### Methods

The first gradient moment of symBIR-8 preparation is given by:

m₁^{symBIR8}=4·G_{MAX}·(F+R)·(F+2R+T_{RF}) (9)

where F is the flat top time and R is the gradient rise time. The RF pulse used 2 ms BIR segments as previously. The response of static spins to symBIR-8 pulse was simulated with time constants 10⁻⁴ s to 1 s with Aₙ=0.25%.

The BIR-4, BIR-8 and symBIR-8 preparations were then evaluated in a phantom. To eliminate the effects of diffusion, an 18 cm spherical silicone oil phantom was used. The phantom was placed at the center of the 32 channel head receive coil and positioned near the magnet isocenter. MR safe sandbags were used to immobilize the phantom. The preparations were applied immediately prior to a spin echo EPI readout without crushers. The TE was 37 ms, FOV=20 cm, 64×64 matrix and slice thickness was 8 mm. These are examples of the parameters only and other parameters may be used. Data were normalized for receive coil sensitivity using the scanner “pre-scan normalize” option. Each preparation (BIR-4, BIR-8 and symBIR-8) was applied with V_{cut}=2 cm/s, with G_{MAX}=10, 20 and 40 mT/m. This was repeated for each tagging direction (X, Y, Z), readout direction (sagittal, transverse and coronal). This resulted in 81 acquisitions in total, with a TR of 3 s and 16 tag and control pairs. The value for M₀ was determined from a scan without a velocity selective preparation with TR=30 s.

### Results

FIG. 5A is a symBIR-8 pulse diagram for V_{cut}=2 cm/s at G_{MAX}=40 mT/m. The simulations show that the symBIR-8 (FIG. 5C) does further reduce eddy current effects compared to the BIR-8 (shown in FIG. 5B).

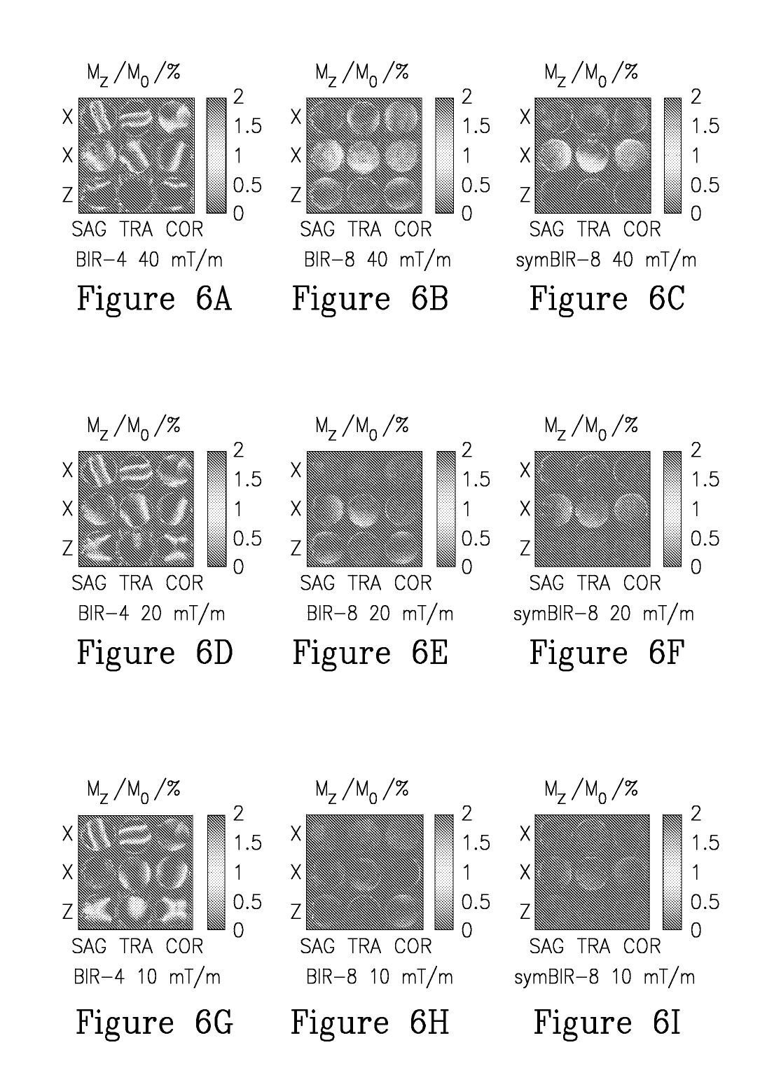

The mean ΔM subtraction images for all three preparations, tagging directions, readout directions and gradient strengths are all depicted in FIG. 6. The BIR-4 (left column) has the greatest amount of artifacts compared to the BIR-8 (middle) and symBIR-8 (right). When labeling on the X axis the greatest variation is in the Y direction for the BIR-4, which matches the previous in vivo data, above. Similarly, when labeling on the Y-axis the variation is along X for the BIR-4. This is not the case for BIR-8 or symBIR-8, where the spatial variation of the artifacts is along the direction of the applied gradient. All artifacts are reduced as G_{MAX} is reduced. Artifactual signal at isocenter is apparent for all preparations. The artifacts are reduced when using symBIR-8 compared to BIR-8, especially on the X and Z labeling axes.

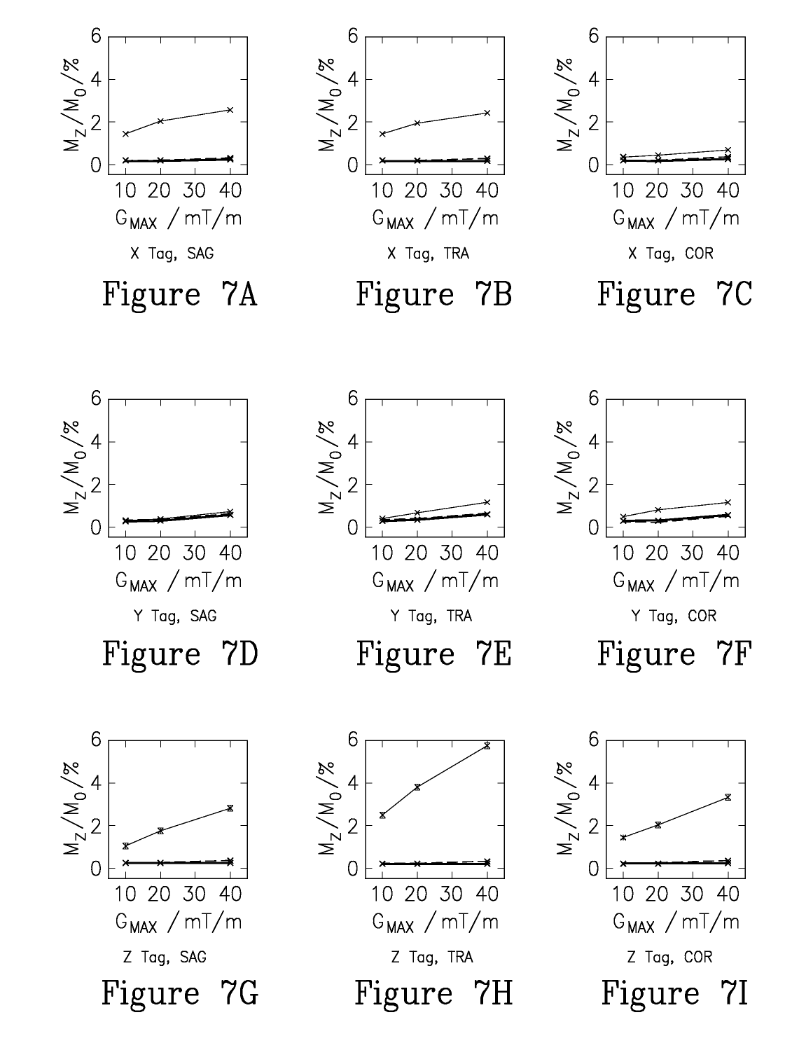

The data in FIG. 6 were quantified by taking the average root mean square error in a mask containing the phantom, plotted in FIG. 7, where the top line in each figure is the quantified subtraction error for BIR-4, the middle line is for BIR-8 and the lowest error line is for symBIR-8. Velocity gradients are applied in X (a-c), Y (d-f) and Z (g-i) directions. Data are from masks of the images in FIG. 6. The root mean squared error is calculated for each tag control pair, data are the mean of this±SD over the 16 tag control pairs.

This phantom experiment confirms that the symBIR-8 preparation does have reduced artifacts compared to the BIR-8, particularly on the X and Y axes. As these artifacts have a special distribution and reduce with G_{MAX}, they are attributed to eddy currents. The artifacts are unlikely to be from diffusion as the diffusion coefficient of the silicone oil is of the order of 1 to 2 orders of magnitude lower than water and the artifacts are spatially inhomogeneous.

There was not a significant difference between symBIR-8 at 20 mT/m to 10 mT/m, so to minimize T₂ decay during the preparation, 20 mT/m was used on the scanner.

Eddy currents during the VS preparation cause unwanted tagging of static tissue and hence an overestimation of perfusion in VSASL. The BIR-8 preparation of the present invention is a highly robust VS preparation to both eddy currents and B₁ with excellent efficiency compared to prior art VS preparations. Its use improves the quality and reliability of VSASL measurements. The symBIR-8 preparation yields even better eddy current results.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1. A velocity selective preparation method, for velocity selective arterial spin labelling (VSASL), said VSASL method using non-selective radiofrequency pulses and magnetic field gradients to modulate the longitudinal magnetization of the spins as a function of their velocity, wherein said velocity selective preparation method comprises an n-segment B₁ insensitive rotation that is resistant to eddy current artifacts by careful arrangement of gradient pulse positions at the zero crossings of the RF pulse waveforms.

2. The method of claim 1 wherein arterial spins are tagged based on their velocity wherein the velocity spin preparations saturate spins above a pre-defined V_{c}, wherein the spins are first tipped into the transverse plane without spatial selection, wherein bipolar gradients are then applied which result in a phase accrual of the spins that is proportional to their velocity, wherein the spins are then flipped back to the longitudinal axis and wherein the longitudinal magnetization of the spins at the end of a VS preparation is then given by the following equation:
M_{Z}(v)=M₀α cos(γm₁v)
where α is the tagging efficiency of the preparation, m₁ is the first moment of the Velocity Selective (VS) gradients and v is the velocity of the spins.

3. The method of claim 2, wherein within a laminar vessel the total expected magnetization is given by the following equation:
$${M}_{z} ( {V}_{MAX} ) = \frac{{M}_{0} α}{{V}_{MAX}} {\int }_{0}^{{V}_{MAX}} cos ( γ {m}_{1} v ) d v = {M}_{0} α sinc ( γ {m}_{1} {V}_{MAX} )$$
where V_{MAX} is the maximum velocity of the vessel, wherein V_{c} is then defined as the first zero crossing of the sin c function, where V_{c}=π/(γm₁).

4. The method of claim 3 wherein V_{c} is in the range 0-100 cm/s.

5. The method of claim 1, wherein the B₁ insensitive rotation is of the order of 4, 8, 16 or more.

6. The method of claim 1, wherein errors due to eddy currents are further reduced by inserting gradient lobes at all four |B1|=0 points of the BIR-8 preparation with polarities −1:+1:+1:−1, giving a symmetric preparation (symBIR-8), and similarly for higher orders of B1 insensitive rotation pulse trains.

7. The method of claim 7 wherein the first gradient moment of the symBIR-8 preparation is given by:
m₁^{symBIR8}=4·G_{MAX}·(F+R)·(F+2R+T_{RF})
where F is the flat top time and R is the gradient rise time.
